(19) United States
(12) Patent Application Publication
(10) Pub. No.: US 2010/0323023 A1
(43) Pub. Date: Dec. 23, 2010

(54) COMPOSITION FOR THE TREATMENT OF A DETACHED RETINA AND METHOD OF PRODUCTION THEREOF

(75) Inventors: Michael Garvey, Wirral (GB); Rachel Lucinda Williams, Neston (GB); David Sai Hung Wong, Wirral (GB)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: ULIVE ENTERPRISES LIMITED, Liverpllo (GB)

(21) Appl. No.: 12/811,566

(22) PCT Filed: Dec. 3, 2008
(86) PCT No.: PCT/GB2008/051148

§ 371 Date: Aug. 19, 2010

(30) Foreign Application Priority Data
Dec. 12, 2007 (GB) 0724226.6

(51) Int. Cl. A61K 9/50; A61K 9/14; A61K 47/34; A61P 27/02; A61K 47/02

(52) U.S. Cl. 424/490; 424/489; 514/772.3; 514/770; 977/773

(57) ABSTRACT

The present invention relates to a composition for use in the treatment of a detached retina, comprising a dispersion of nanoparticles in a liquid, wherein the nanoparticles have a specific gravity which is higher than that of the liquid and the individual refractive indices of the nanoparticles and the liquid are substantially similar to one another. The present invention also relates to a composition for use in the treatment of a detached retina, comprising a dispersion of silica nanoparticles in a liquid, a method and kit of parts for producing the compositions and a method of treating a detached retina.

The present invention relates to a composition for use in the treatment of retinal detachment. In particular, the invention is concerned with providing a “heavy” retinal tamponade for use in the treatment of a detached retina in the lower part of an eye.

Retinal detachment is the separation of the neurosensory retina from its underlying pigment epithelium. Untreated, retinal detachment can result in permanent vision loss or blindness. Retinal detachment is caused by traction of the vitreous upon the retina. The traction can be ‘dynamic’, caused by eye movements and thus relative movement of the vitreous and the retina; or ‘static’, due to contraction of membranes on the surface of the retina. Retinal detachments are associated with myopia, pseudophakia, trauma, diabetes and are often the common pathway leading to blindness in a host of eye diseases.

Where a retinal detachment is associated with retinal breaks (also referred to as perforations, holes or tears), fluid gains access from the vitreous cavity to the subretinal space. This form of retinal detachment is referred to as ‘Rhegmatogenous’. There are several effective means of closing retinal breaks. The first involves the application of explants outside the eye in order to buckle the sclera (such as described in U.S. Pat. No. 6,547,714). The second involves the use of internal tamponades. Internal tamponades are agents injected into the vitreous cavity to occlude retinal breaks. They are fluids that are immiscible with water and form an interface with it. The fluid can be gaseous such as air, sulphur hexafluoride (SF₆) or perfluoropropane (C₃F₈). These gases can be used undiluted in small volumes or mixed with air and totally fill the vitreous cavity. The liquids include perfluorocarbon liquids, semifluorinated alkanes or alkenes and silicone oil. Of these, only silicone oil can be tolerated in the eye for more than a few weeks. Prolonged use of any of the other liquids will give rise to retinal toxicity as demonstrated by inflammatory reaction or by histological changes.

Retinal detachments can also be treated by means of pneumatic retinopexy, whereby a gas bubble is injected into the vitreous space so as to help push the retinal tear back against the wall of the eye. This method can also be used in conjunction with the laser and cryo-surgical techniques if required. The gases preferred for such operations are commonly either perfluoropropane (C₃F₈) or sulphur hexafluoride (SF₆), which when mixed with sterile air have the properties of remaining in the eye for extended periods of time. RU2235527 discloses a number of other gases that may also be used in conjunction with this technique. Eventually, the gas is replaced by the eyes own natural fluid. There have been recent concerns over the toxicology of compositions that are fluorine based.

Another method of treatment involves a vitrectomy whereby all or part of the vitreous gel is removed from the eye and replaced with a tamponade agent, such as a perfluorocarbon liquid, silicone oil or a gas (using a similar gaseous composition as described above). Following removal of the liquid or resorption of the gas, the eye is allowed to fill with the body's own fluid over time. In this technique, a small incision is made in the wall of the eye and the vitreous gel is removed by means of a small cutting device. As the vitreous gel is removed, a saline solution is used to maintain the pressure by a continuous infusion. This solution is then exchanged with an air infusion following which an air and gas mixture is injected. Alternatively, perfluorocarbon liquids, semifluorinated alkanes or alkenes and more commonly silicone oil is injected as a tamponade agent. The tamponade agent is the immiscible fluid that occludes retinal breaks because of its interfacial tension and its buoyancy. The tamponade material is therefore intended to close the retinal tear and reoppose the retina on the underlying choroids.

Whilst these methods of treatment can be successful, they often require multiple treatments and there is always a danger of physical damage to the retina itself. It is particularly difficult to treat retinal detachment in the lower part of the eye as the longer term tamponade agents, eg silicone oil, or the gases have a lower specific gravity than the body's own fluid and therefore float on top of the remaining aqueous in the eye.

In order to address the problem associated with retinal detachment in the lower part of the eye, a number of “heavy” tamponades have been developed. For example, Wong and colleagues (Wong et al., (2006) Ann. Acad. Med. Singapore, (3) 181-184) have employed the used of Densiron®-68 (Fluoron, Germany) as a heavier-than-water tamponade. Densiron®-68 is a homogeneous mixture of perfluorohexyloctane and conventional silicone oil.

In recent years, the use of magnetic particles has been developed. ES2132029 and ES2024242 disclose using biocompatible magnetic particles which are suspended in a biocompatible viscoelastic substance and which are attracted to a magnet located on the sclera adjacent to the detached retina. U.S. Pat. No. 6,547,714; U.S. Pat. No. 6,135,118; and U.S. Pat. No. 6,464,968 describe inserting a magnetic fluid in a carrier of dimethyl-siloxane into an eye and applying a magnetic field to the fluid by means of a magnetized scleral buckle. Both techniques may result in damage to the sclera.

It is an object of the present invention to address one or more problems associated with the prior art procedures and in particular to provide a tamponade agent that can be effectively used in the treatment of retinal detachment. Furthermore, it is an object of the present invention to provide a tamponade agent that can be used to treat retinal detachment of the lower part of the eye.

In accordance with the present invention, there is provided a composition for use in the treatment of a detached retina, comprising a dispersion of nanoparticles in a liquid, wherein the nanoparticles have a specific gravity which is higher than that of the liquid and the individual refractive indices of the nanoparticles and the liquid are substantially similar to one another.

The concentration of the nanoparticles in the liquid is preferably in the range of 0.1 to 30% by weight. More preferably, the concentration of the nanoparticles in the liquid is in the range of 0.1 to 20% by weight. Most preferably, the concentration of the nanoparticles in the liquid is in the range of 0.1 to 10% by weight.

The term “substantially similar” is intended to mean that the refractive indices of both the nanoparticles and liquid are selected to be as similar as possible, for example within ±10%. It will be apparent to the skilled addressee that the more similar the refractive indices are to one another, the less optical turbidity is present in the composition. Preferably, the individual refractive indices of the nanoparticles and the liquid are within 0.5 of one another. More preferably, the refractive indices of the nanoparticles and the liquid are within 0.05 of one another. If desired the liquid may comprise two or more liquids mixed together so as to form a liquid having a substantially similar refractive index to that of the nanoparticles.

The individual refractive indices of the nanoparticles and the liquid will preferably have a lower limit of 1.2. More preferably, the individual refractive indices will have a lower limit of 1.3. Most preferably, the individual refractive indices will have a lower limit of 1.4. The individual refractive indices of the nanoparticles and the liquid will preferably have an upper limit of 1.8. More preferably, the individual refractive indices will have an upper limit of 1.7. Most preferably, the individual refractive indices will have an upper limit of 1.6. A range of individual refractive indices of the nanoparticles and the liquid may be selected from any one of the upper or lower limits as herein above described (e.g. 1.2 to 1.8, 1.3 to 1.7 and 1.4 to 1.6). In the most preferred embodiment, the individual refractive indices of the nanoparticles and the liquid are in the region of 1.5±0.05.

The composition can be used to replace part or all of the vitreous gel in the posterior part of the eye. The inclusion of the nanoparticles increases the specific gravity of the liquid allowing the composition to be used as a “heavy” retinal tamponade to repair detached retinas in the lower half of the eye.

The dispersion of nanoparticles in the liquid can be accomplished by any suitable method. By way of non-limiting example, ultrasonic mixing can be used to form the dispersion of nanoparticles in the liquid. This type of mixing can allow a greater amount of nanoparticles to be dispersed in the liquid and therefore a higher specific gravity composition to be achieved whilst retaining a low shear viscosity.

In some cases, therefore, the concentration by weight of the nanoparticles in the liquid may be relatively high, for example up to 30%, e.g. 5 to 18% or 10 to 15%.

Preferably, the nanoparticles comprise silica particles.

In accordance with a further aspect of the present invention, there is provided a composition for use in the treatment of a detached retina, comprising a dispersion of silica nanoparticles in a liquid.

The nanoparticles for both aspects of the above aspects will preferably share a number of characteristics listed below.

The silica nanoparticles may be amorphous. It is preferred that the silica particles comprise fumed silica—such as the fumed silica marketed under the name Aerosil®380 by Degussa-Silanes. Silica has refractive index (depending upon source and form) between 1.4-1.55.

The nanoparticles may take the form of matrix micro beads, and/or beads which carry other agents, moieties, molecules or compounds (e.g. drugs, bioactive molecules or other beneficial or useful entities). Thus, the particles may comprise gelled or aggregated nanoparticles (for example gelled or aggregated silica nanoparticles). The particles may contain or carry the other agents by various mechanisms including adsorption, absorption or entrapment, particularly when they comprise gelled or aggregated nanoparticles (for example gelled or aggregated silica nanoparticles).

The nanoparticles may comprise nanoparticles of similar sizes to one another. Alternatively, the nanoparticles may comprise nanoparticles of different sizes. Preferably, the size of the nanoparticles is less than 100 nm. More preferably, the size of the nanoparticles is less than 50 nm. It will be apparent, to the skilled addressee, that the concentration of nanoparticles used will be determined by a number of factors, such as the required specific gravity (which may be dependent upon location and size of the retinal detachment).

The liquid may comprise one or more oils. Preferably, the liquid comprises a silicone oil. One type of silicone oil has been shown to have a specific gravity of 0.97 g/cm³ (Wetterqvist et al., 2004, Br. J. Opthalmol, 88, 692-6). The liquid may comprise a mixture of dimethyl- and diphenyl- siloxanes, for example, (85-88%)Dimethyl-(12-15%)diphenylsiloxane. (85-88%)Dimethyl-(12-15%)diphenylsiloxane has a refractive index of 1.479 (CAS#68083-14-7 obtained from Fluorochem Limited, UK).

If required, the composition may further comprise an additive to improve the rheological properties of the fluid. Additives that will not impair the optical characteristics of the composition are most desirable.

Whilst the liquid used in accordance with the present invention may be one which is currently used during the treatment of retinal detachment, it will be evident to the skilled addressee that the term “liquid” will also encompass liquids which have yet to be developed.

In accordance with a further aspect of the present invention, there is provided a method of producing a composition for use in the treatment of a detached retina, the method comprising mixing a plurality of nanoparticles with a liquid, wherein the nanoparticles have a specific gravity which is higher than that of the liquid and the individual refractive indices of the nanoparticles and the liquid are substantially similar to one another.

In accordance with a yet further aspect of the present invention, there is provided a method of producing a composition for use in the treatment of a detached retina, the method comprising mixing a plurality of silica particles with a liquid so as to form a dispersion.

Both methods may be used to produce compositions as herein above described.

In accordance with yet another aspect of the present invention, there is provided a kit of parts for producing a composition for use in the treatment of a detached retina comprising,
a) a liquid;
b) a plurality of nanoparticles having a specific gravity which is higher than the liquid and having a refractive index which is substantially similar to that of the liquid; and
c) means to disperse the nanoparticles in the liquid.

In accordance with a further aspect of the present invention, there is provided a kit of parts for producing a composition for use in the treatment of a detached retina comprising,
a) a liquid;
b) a plurality of silica nanoparticles; and
c) means to disperse the nanoparticles in the liquid.

Preferably, both kit of parts may be used to produce compositions as herein above described.

The kit of parts may further comprise a means by which to measure a given amount of the nanoparticles and liquid prior to use.

In accordance with a further aspect of the present invention, there is provided a method of treating a detached retina comprising the steps:
a) removing at least part of the vitreous humour and/or other fluid from a location near to or adjacent to the area of retinal detachment; and
b) replacing the removed humour and/or other fluid with a composition comprising a dispersion of nanoparticles in a liquid.

It will be apparent to the skilled addressee that the method of treating a detached retina will be compatible with existing methods of eye surgery. For example, the composition may be inserted into the eye after or during a vitrectomy and by means of standard operating equipment. The nanoparticles increase the specific gravity of the liquid, permitting the surgeon to use the liquid to give prolonged endotamponade to the lower fundus.

Preferably, the method of treatment utilises compositions as herein described above.

The present invention will now be more particularly described by way of example only with reference to the following examples:

### EXAMPLE 1

A composition for use as a heavier-than-water tamponade was prepared by dispersing 0.3 g of Aerosil® R972 (Degussa GmbH, Germany) in 9.9 g of (85-88%)Dimethyl-(12-15%)diphenylsiloxane copolymer (CAS#68083-14-7 obtained from Fluorochem Limited, UK). The dispersion was placed on a roller mixer and mixed until the silica particles were thoroughly dispersed throughout the siloxane copolymer and an optically clear composition formed. The refractive index of the Aerosil® silica particles will be in the region of 1.44 to 1.48 which is substantially similar to refractive index of 1.479 of the siloxane copolymer.

### EXAMPLE 2

An experiment was also undertaken using a polydimethylsiloxane oil in place of the siloxane copolymer. The refractive index of polydimethylsiloxane oil is approximately 1.4. The composition produced had a degree of turbidity, but could still be utilised as a heavy retinal tamponade. It will be apparent that a degree of turbidity will be acceptable in the treatment of a detached retina, as the tamponade may be temporarily introduced into the eye in order to restore the long term sight of an individual.

### EXAMPLE 3

A composition containing 11.0% w/w silica for use as a heavier-than-water tamponade was prepared by adding 9.6 g Aerosil R972 Pharma silica (ex Evonik) to 78.2 g of phenyltrimethicone oil (556 Cosmetic Grade Fluid ex Dow Corning). The composition was placed on a roller mixer at ambient temperature for ca. 48 hours i.e. until the silica was fully dispersed throughout the oil to form an optically clear dispersion. The Refractive Index of the silica particles will be in the region 1.44 to 1.48 which is substantially similar to the refractive index of the oil (1.46).

### EXAMPLE 4

Three compositions for use as heavier-than-water tamponades containing 10.0% w/w, 12.5% w/w and 15.0% w/w Aerosil R972 Pharma silica (ex Evonik) in phenyltrimethicone oil (556 Cosmetic Grade Fluid ex Dow Corning) were prepared respectively as follows. 3.0 g Aerosil R972 Pharma silica was added to 27.0 g phenyltrimethicone oil; 3.75 g Aerosil R972 Pharma silica was added to 26.25 g phenyltrimethicone oil; 4.5 g Aerosil R972 Pharma silica was added to 25.5 g phenyltrimethicone oil. Each composition was placed on a roller mixer for 10 days at ambient temperature after which in each case the silica was fully dispersed throughout the oil to form an optically clear dispersion. The shear viscosity of each blend was measured using a TA Instruments Advanced Rotational AR500 Rheometer. In addition, 5 g aliquots of each composition were ultrasonically mixed using a Misonix Microson ultrasonic probe (each aliquot being mixed for 2 minutes at a power setting of 10 Watts). The shear viscosity of the ultrasonically mixed aliquots was measured using the AR500 Rheometer. Shear viscosity data for the roller-mixed only compositions and the ultrasonically mixed aliquots are given below.

| | | Shear Viscosity (Pas) | | | | |
|---|---|---|---|---|---|---|
| | | | | | Maximum | |
| [silica] w/w | Appearance | 10 s−1 | 25 s−1 | 100 s−1 | viscosity | |
| 10.0% | Clear; | 11.84 | 10.28 | 3.285 | 12.02 | (15.86 s⁻¹) |
| | silica fully dispersed | | | | | |
| 10.0% - | As above (except lower | 0.0925 | 0.0897 | 0.1026 | 0.454 | (0.10 s⁻¹) |
| ultrasonically | viscosity); no apparent | | | | | |
| mixed | settling | | | | | |
| 12.5% | Clear | 62.41 | 22.46 | 1.756 | 107.4 | (2.51 s⁻¹) |
| | silica fully dispersed | | | | | |
| 12.5% - | As above (except lower | 0.1371 | 0.1304 | 0.1748 | 0.3726 | (0.10 s⁻¹) |
| ultrasonically | viscosity); no apparent | | | | | |
| mixed | settling | | | | | |
| 15.0% | Clear | 125.4 | 47.6 | 1.407 | 1654 | (0.11 s⁻¹) |
| | silica fully dispersed | | | | | |
| 15.0% - | As above (except lower | 0.1934 | 0.2019 | 0.6196 | 1.03 | (398.1 s⁻¹) |
| ultrasonically | viscosity); very small | | | | | |
| mixed | amount of settling after 1 | | | | | |
| | day at ambient temperature | | | | | |
| 556 Cosmetic | Clear | 0.0202 | 0.0213 | 0.0213 | 0.352 | (0.1 s⁻¹) |
| Grade Fluid | | | | | | |
| (control) | | | | | | |

For all three compositions ultrasonic mixing produced a large decrease in shear viscosity. It is evident that this type of mixing allows a higher amount of silica to be dispersed in the silicone oil and therefore a higher composition specific gravity to be achieved whilst retaining a relatively low shear viscosity.

1. A composition for use in the treatment of a detached retina, comprising a dispersion of nanoparticles in a liquid, wherein the nanoparticles have a specific gravity which is higher than that of the liquid and the individual refractive indices of the nanoparticles and the liquid are substantially similar to one another.

2. A composition as claimed in claim 1, wherein the concentration of the nanoparticles in the liquid is in the range of 0.1 to 30% by weight.

3. A composition as claimed in either claim 1 or 2, wherein the individual refractive indices of the nanoparticles and the liquid are in the range of 1.4 to 1.6.

4. A composition as claimed in any preceding claim, wherein the individual refractive indices of the nanoparticles and the liquid are within 0.5 of one another.

5. A composition as claimed in any preceding claim, wherein the refractive indices of the nanoparticles and the liquid are within 0.05 of one another.

6. A composition as claimed in any preceding claim, wherein the nanoparticles comprise silica particles.

7. A composition for use in the treatment of a detached retina, comprising a dispersion of silica nanoparticles in a liquid.

8. A composition as claimed in any preceding claim, wherein the nanoparticles are less than 100 nm in size.

9. A composition as claimed in any preceding claim, wherein the nanoparticles are less than 50 nm in size.

10. A composition as claimed in any one of claims 6 to 9, wherein the liquid comprises silicone.

11. A composition as claimed in any one of claims 6 to 10, wherein the silica nanoparticles are amorphous.

12. A composition as claimed in any one of claims 6 to 11, wherein the silica particles comprise fumed silica.

13. A composition as claimed in any one of claims 6 to 12, wherein the silica particles comprise gelled or aggregated silica nanoparticles.

14. A composition as claimed in any one of claims 6 to 13, wherein the silica nanoparticles contain adsorbed, absorbed or entrapped drugs or other bioactive molecules.

15. A composition as claimed in any preceding claim, wherein the liquid comprises one or more oils.

16. A composition as claimed in any preceding claim, wherein the liquid comprises a siloxane oil.

17. A composition as claimed in any preceding claim, wherein the liquid comprises a functionalized siloxane.

18. A composition as claimed in any preceding claim, wherein the liquid comprises a methyl siloxane.

19. A composition as claimed in any preceding claim, wherein the liquid comprises a phenyl siloxane.

20. A composition as claimed in any preceding claim, wherein the liquid comprises a mixture of methyl and phenyl siloxanes or phenylated methyl siloxanes.

21. A composition as claimed in any preceding claim, wherein the liquid comprises trimethylphenylsiloxane.

22. A composition as claimed in any preceding claim, wherein the liquid comprises a mixture or copolymer of dimethyl- and diphenylsiloxanes.

23. A composition as claimed in claim 22, wherein the liquid comprises a copolymer of 85-88% dimethylsiloxane and 12-15% diphenylsiloxane.

24. A composition as claimed in any preceding claim, wherein the liquid comprises phenyltrimethicone.

25. A composition as claimed in any preceding claim, wherein the composition further comprises an additive to improve the rheological properties of the liquid.

26. A method of producing a composition for use in the treatment of a detached retina, the method comprising mixing a plurality of nanoparticles with a liquid, wherein the nanoparticles have a specific gravity which is higher than that of the liquid and the individual refractive indices of the nanoparticles and the liquid are substantially similar to one another.

27. A method as claimed in claim 26 wherein the mixing is carried out ultrasonically.

28. A method as claimed in claim 26 or claim 27, wherein the method is used to produce a composition as claimed in any one of claims 1 to 25.

29. A method of producing a composition for use in the treatment of a detached retina, the method comprising mixing a plurality of silica particles with a liquid so as to form a dispersion.

30. A method as claimed in claim 29 wherein the mixing is carried out ultrasonically.

31. A method as claimed in claim 29 or claim 30, wherein the method is used to produce a composition as claimed in any one of claims 7 to 25.

32. A kit of parts for producing a composition for use in the treatment of a detached retina comprising,
a) a liquid;
b) a plurality of nanoparticles having a specific gravity which is higher than the liquid and having a refractive index which is substantially similar to that of the liquid; and
c) means to disperse the nanoparticles in the liquid.

33. A kit of parts as claimed in claim 32, wherein the kit of parts is used to produce a composition as claimed in any of claims 1 to 25.

34. A kit of parts for producing a composition for use in the treatment of a detached retina comprising,
a) a liquid;
b) a plurality of silica nanoparticles; and
c) means to disperse the nanoparticles in the liquid.

35. A kit of parts as claimed in claim 34, wherein the kit of parts is used to produce a composition as claimed in any of claims 7 to 25.

36. A kit of parts as claimed in any one of claims 32 to 35, wherein the kit of parts further comprises a means by which to measure a given amount of the nanoparticles and liquid prior to dispersal.

37. A method of treating a detached retina comprising the steps:
a) removing at least part of the vitreous humour and/or other fluid from a location near to or adjacent to the area of retinal detachment; and
b) replacing the removed humour and/or other fluid with a composition comprising a dispersion of nanoparticles in a liquid

38. A method as claimed in claim 37, wherein the composition comprises a composition as claimed in any one of claims 1 to 25.
